(19) United States
(12) Patent Application Publication
(10) Pub. No.: US 2025/0331829 A1
(43) Pub. Date: Oct. 30, 2025

(54) Robotic Pill System for Active Sample Collection and Related Method

(71) Applicant: The Board of Trustees of the Leland Stanford Junior University, Stanford, CA (US)

(72) Inventors: Victor Julian Garcia Gradilla, San Diego, CA (US); Fernando Soto, Millbrae, CA (US); Demir Akin, Palo Alto, CA (US); Utkan Demirci, Stanford, CA (US)

(21) Appl. No.: 19/194,867

(22) Filed: Apr. 30, 2025

Related U.S. Application Data
(60) Provisional application No. 63/640,778, filed on Apr. 30, 2024.

(51) Int. Cl. A61B 10/00

(52) U.S. Cl. CPC A61B 10/0045; A61B 2010/0061

(57) ABSTRACT

A robotic pill system for collecting a sample from human and animal body cavities and liquid resources using a robotic pill. The system includes a robotic pill adapted to collect the sample when the robotic pill is received or introduced or taken through an orifice, other natural or surgical openings and cavity. A screw is present in the housing of the robotic pill and is adapted to collect the sample(s) from an opening in the housing into one or more collection chambers in the housing of the robotic pill. The robotic pill may be positionally located before operation of a sample as well as its position directed and may utilize a wireless interface to transmit positional information and/or control information for the screw.

### CROSS-REFERENCE TO RELATED APPLICATION

This application claims the benefit of U.S. Provisional Patent Application No. 63/640,778 entitled “G-I-NTELLIGENT PILL FOR ACTIVE SAMPLE COLLECTION IN THE GASTROINTESTINAL TRACT” filed Apr. 30, 2024, the contents of which are incorporated by reference herein in its entirety for all purposes.

### STATEMENT OF FEDERALLY SPONSORED RESEARCH OR DEVELOPMENT

This invention was made with Government support under contract R21AG072675 awarded by the National Institutes of Health. The Government has certain rights in the invention.

### TECHNICAL FIELD

This disclosure relates to the collection of samples, including but not limited to viscous mucosal samples from the gastrointestinal tract, utilizing a robotic pill device.

### BACKGROUND

Neuropsychiatric disorders are a widespread and debilitating condition affecting over 100 million Americans. They are responsible for disability and incur significant healthcare and societal cost. Costs related to neurological disease are expected to reach $600 billion by 2030 due to the aging population. Recent studies have shown that some neuropsychiatric disorders can be caused by altered gut-brain signaling. The vagus nerve connects the brain to the small intestine and plays a role in transmitting sensory information and bioanalyte signals. For example, 90% of serotonin produced by the body is produced by bacteria in the gastrointestinal tract (GIT). However, monitoring neurologically relevant markers in the GIT is challenging, as it often requires invasive procedures that yield poor data. The GIT is over 9 meters in total length and comprises multiple sections with unique characteristics. Even within the same section, target markers of disease can be localized to a small microenvironment. Thus, collecting medically relevant bioanalytes from the GIT to monitor health and disease in a non-invasive, cost-effective manner remains challenging, often requiring invasive procedures like colonoscopies and imaging techniques that are not easily accessible for routine screening or repeat measurements or carry risks associated with surgical/interventional procedures in a hospital setting. The result is incomplete, infrequent, or poor-quality data that is not medically actionable. Moreover, liquid biopsies, such as blood, urine, or stool samples, now important in identifying health concerns, cannot reveal the specific location of origin of bioanalytes.

Gastrointestinal (GI) mucosa is a viscous coating layer that surrounds the interior surface of the gastrointestinal tract. It is the first barrier in the stomach wall and between the lumen and intestines. Some of its functions are related with digestion, absorbance of nutrients and as a defense using physical mechanisms and specific immunological responses against bacteria and toxic agents. Thus, the mucus layer contains a matrix of biofluidic substances including antibodies, biomarkers, genetic materials, peptides, enzymes, amino acids, bacteria, viruses, and so forth, with important information that might be used for detection, study, and diagnosis of different diseases at early stages. However, the viscous mechanical properties of mucus make it difficult to sample inside the tract. Available techniques such as colonoscopies and invasive surgical procedures used for diagnosis are usually deployed at advanced stages, are expensive, and are not suitable for frequent or routine use.

### SUMMARY

The collection of viscous samples, such as mucus, remains a substantial challenge due to the complex mechanical properties and non-Newtonian behavior of the mucus.

While so-called smart pills have been used in diagnostics, biosensing, imaging, and drug delivery, current smart pills have been limited in their ability to collect viscous samples, such a mucus. This is, in part, because current smart pills for the gastrointestinal tract using physical absorption, one-way valves and osmotic gradients are limited to collection of low or non-viscous fluids. These pills have enabled various sampling approaches but primarily rely on passive diffusion of the target samples into the pill. Accordingly, while some smart pills have been used for GIT sampling, those previous pills and methods are limited in their ability to assess bioanalyte concentration, distribution, and changes in different regions of the GIT. A non-invasive, cost-effective method is therefore needed for collecting bioanalytes from the GIT particularly in viscous samples.

A novel and cost-effective tool is proposed herein to provide insight for precision medicine and improve early disease detection and health monitoring through sequential temporal isolation of bioanalytes in specified GIT locations utilizing a robotic pill. The ability to generate comprehensive datasets could shed light on the transition from health to disease and vice versa.

This extraction and analysis of GI mucus biofluids may extend the understanding of the gastrointestinal tract including the corresponding diseases and pathologies. The robotic pill described herein is capable of capturing gastrointestinal mucosa and microenvironmental fluids and may facilitate spatial and temporal sampling, offering more insights into gut-brain signaling and its function in relation to neurodevelopmental disorders. It is hypothesized that the intestinal mucosal layer contains localized signatures that may provide added information not obtainable through traditional liquid biopsy methods. Therefore, this disclosure proposes to address these limitations in a next-generation smart sampling pill or robotic pill. This disclosure presents an approach to achieve the untethered active capture of viscous mucosa sample for its use in the GI tract. It should be appreciated that the sample may include mucus, but it is not so limited. Techniques and devices are presented herein for the collection of viscous fluids, along with liquids and solids or a combination of them in the gastrointestinal tract. This means the sample can or could potentially include mucus, liquids, and/or hard materials such as digested and/or undigested food.

In some preferred exemplary forms, such a robotic pill may be magnetically actuated and include a motorized screw for active collection of viscous samples such as mucus, overcoming passive diffusion limitations, into one or more collection chambers in the robotic pill which can be detachable from the pill for recovery of the sample. In some forms, the pill may further leverage a three-axis sensor (such as a Hall effect sensor) and/or wireless communications (such as BLE communications) to positionally locate and control the robotic pill. Such a robotic pill may facilitate minimally invasive and precise sampling. It is seen as having potential applications in targeted biomarker discovery and early disease detection and could transform diagnostics for hard-to-reach regions like the gastrointestinal tract with higher specificity due to a proximity advantage.

According to one aspect, a robotic pill system for collecting one or more samples from human and animal body cavities and liquid resources. The robotic pill system includes a robotic pill adapted to collect the one or more samples when the robotic pill is received or introduced or taken through an orifice, other natural or surgical openings, or a cavity of the subject. The robotic pill includes a housing having an opening placing an inside of the housing in fluid communication with the surrounding environment, a screw present in the housing, a motor to drive the screw, and one or more collection chambers. The screw is adapted to collect the one or more samples from the opening in the housing by rotation of the screw by the motor for delivery of the one or more samples into one or more collection chambers in the housing of the robotic pill.

In some forms, the one or more collection chambers may be selectable for use in collection of the one or more samples with the screw in order to provide for the collection of various samples in a spatiotemporally-controlled manner.

In some forms, the robotic pill system may further include an external computing device. The robotic pill may be wirelessly in communication with the external computing device for at least one of control of the robotic pill and collection of data from the robotic pill (or both).

In some forms, the robotic pill system may further include an external magnet apart from the robotic pill. The robotic pill may further include one or more magnets coupled to the robotic pill to facilitate a magnetic actuation and docking of the robotic pill with the external magnet with the external magnet being physically spaced from the robotic pill and not part of the robotic pill.

In some forms, the robotic pill system may further include a three-axis Hall effect magnetic sensor, in which the three-axis Hall effect magnetic sensor is configured to measure a magnitude of a magnetic field along three axes, which varies with the separation distance from the magnet, to spatially locate the robotic pill. The three-axis Hall effect magnetic sensor may be physically coupled and integrated into the robotic pill. It is contemplated in such case, the robotic pill may be wirelessly in communication with an external computing device to transmit such positional information, which may also be validated or supported by other modalities (e.g., imaging of the pill in the subject, through collected pH information from the pull, and so forth).

In some forms, the one or more samples include mucosal samples. Such samples generally could be, but are not limited to, biological samples, viscous materials (such as the aforementioned mucosa), fluid samples, solid samples, and mixes thereof.

In some forms, the robotic pill may further include a control electronic board in electrical communication with at least the motor. The control electronic board may include a wireless communication interface that is in wireless communication with an external computing device. For example, there could be Bluetooth communication between the robotic pill and the external computing device (such as, for example, a mobile device). In some instances, repeaters may be used to help transmit signals to and from the robotic pill. The control electronic board may be configured to transmit to the external computing device via the wireless communication interface information relating to a spatial positioning of the robotic pill and the control electronic board may be configured to receive via the wireless communication interface instructions relating to an operation of the screw by the motor (with the board directing the operation of the motor accordingly).

According to another aspect, a method of operating the robotic pill system as described above is disclosed, which operation occurs after the robotic pill received or introduced or taken through an orifice, other natural or surgical openings, or a cavity of the subject. The method includes, at a minimum, operating the screw to transport the sample through an opening in the housing and into the one or more collection chambers to collect the sample.

In some forms, the method may further involve, before operating the screw, positioning the robotic pill at an operation location (i.e., a location at which the screw is intended to be operated for the collection of a sample) within the subject. Positioning the robotic pill at the operation location within the subject may involve, in some forms of the method, directing the location of the robotic pill by magnetically manipulating the position of the robotic pill to the operation location. Positioning the robotic pill at the operation location within the subject may involve, in some forms, magnetically docking the pill with an external magnet to hold the robotic pill in the operation location while collection occurs. Before operating the screw, the method may involve using a 3-axis Hall effect sensor to spatially locate the robotic pill. Other method and modalities, alternatively or in conjunction with one another, may also be used to position and validate the position of the robotic pill prior to operation.

In some forms of the method, operating the screw to transport the sample through an opening in the housing and into the one or more collection chambers may involve the robotic pill receiving instructions via a wireless communication interface to control operation of the screw by the motor (and then acting on those instructions by operating the motor).

In some forms of the method, the sample may be a mucosal sample or another viscous sample whose collection is particularly well facilitated by the screw (and without which screw, the collection may be challenging based on the comparative viscosity to other less-viscous samples). Again, the robotic pill incorporating a driven screw is not so limited to just viscous sample collection but is well suited for it.

In some forms of the method, after the sample is collected, the robotic pill may be removed or otherwise exerted from the subject and recovered. The method may then further include the step of recovering the sample from the robotic pill. Ater recovering the sample from the robotic pill, an analysis of the collected sample may be performed.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE FIGURES

FIG. 1 illustrates an exemplary robotic pill for active viscous sample collection. FIG. 1, upper left panel, is an actual picture of the robotic pill device. FIG. 1, upper right panel, illustrates the robotic pill device disassembled. The left and right lower panels include isometric views of the robotic pill device assembled and disassembled and identify some of the components by name with lead lines.

FIG. 2 depicts the general use case and a capsule screw conveyor mechanism for the robotic pill engaging a mucus layer of the GIT. The pill can be introduced in the GI tract by ingestion, travel through different sections of the GI tract for sample collection and extracted by excretion for subsequent downstream analysis of the collected analytes. In right upper panels of FIG. 2, a screw conveyor mechanism is shown for collection and transport of viscous biological sample into the storage chamber/compartment of the robotic pill.

FIG. 3 shows in greater detail an exemplary capsule or robotic pill with multiple containers for multipoint sampling. In upper panel A of FIG. 3, the robotic pill components are illustrated including a pill rounded shape polymer biocompatible case (1), a biosafe battery (2), micromotors (3), control electronics board (4), a hydrodynamic screw (5), a screw guide housing (6), a rotary chamber with multiple isolated compartments (7a, 7b, and 7c), and positioning magnets (8). In lower panel B of FIG. 3, the assembled multi sampling robotic pill capsule is illustrated. It is contemplated that in some forms, the number of motors can be limited by the use of a gearbox, enabling selection and control the hydrodynamic screw and rotary chamber with a single actuator. The pill contains a NdFeB N52 magnet embedded in both ends of the bottom of the robotic pill. An external magnetic field can be used to dock the pill against the intestine lining. This approach is an alternative to clamping or use of adhesives enabling rapid capture and release of the pill.

FIGS. 4A-4E illustrate and detail the docking behavior of the robotic pill. In FIG. 4A, a scheme of attraction forces is depicted between the magnets in the robotic pill and external magnets. In FIG. 4B, the reorientation in absence and presence of external magnetic field is illustrated (this view also shows the window or aperture for the screw). In FIG. 4C, a docking configuration through a wall is illustrated schematically in the left panel and experimentally in the center and right panels demonstrating the attractive forces between the magnets in the pill and external magnets through an exemplary wall of poly(methyl methacrylate) (PMMA). In FIG. 4D, a graph illustrates the docking force at different distances from the external magnet (1 Tesla, Neodymium magnet). In FIG. 4E, a graph provides the docking force within various barriers made of different materials, each barrier having a 25-mm thickness (n=5, technical replicates for each material).

FIGS. 5A-5G illustrate the magnetic motion control of the exemplary robotic pill. FIGS. 5A-5C provide timelapse images depicting magnetic guidance and motion control of the robotic pill towards the sample location and sample capture. FIG. 5D depicts the robotic pill after sample capture. FIG. 5E shows the disassembly and open view of the collection chamber of the robotic pill after sampling, with the detachable and disposable collection chamber containing the captured sample being removed. FIG. 5F shows gel viscosities at varying dilutions (v:v) as a function of shear rates. The diluted gel exhibited shear-thinning property where viscosities decreased with increased shear rates. FIG. 5G is a graph providing the volume captured at different viscosities (gel dilution), sampling time 60 seconds (n=4, technical replicates for each dilution).

FIGS. 6A-6G illustrate the capture of a biological sample by the robotic pill through a bleeding re-creation in phantom tissue. FIGS. 6A-6C show a progressing timelapse sequence of hemoglobin (Hb) capture in viscous gel by the robotic pill, simulating a potential use case for “bleeding” within a mock sample. FIG. 6D shows the robotic pill after sample capture. FIG. 6E shows the storage chamber disassembled with the captured sample (Hb and phantom tissue). FIGS. 6F and 6G illustrate the detection of captured hemoglobin via spectrophotometry using a Hb Guaiac oxidation colorimetric assay.

FIGS. 7A and 7B illustrate the capture of bovine serum albumin (BSA) in mock gel sample by the robotic pill and its subsequent detection/quantification.

FIG. 8 illustrates the pill electronics architecture in which the upper left panel A shows a sideview of the electronic board indicating the different components, in which the lower left panel of A depicts a block diagram of the electronic system, and the panel B shows the top and bottom views of the actual control electronic board.

FIGS. 9A-9C illustrate the wireless monitoring and activation control of the robotic pill via Bluetooth using a smartphone. FIG. 9A is a schematic representation of the wireless system involving the integration of wireless Bluetooth Low Energy (BLE) communication for sampling activation/deactivation control. FIG. 9B is an actual image of a wireless capture activation of the robotic pill. FIG. 9C is a graph depicting a wireless BLE signal attenuation through air and a 25-mm thick tissue.

FIGS. 10A-10D depict the magnetic location by a 3-axis (3-D) Hall effect magnetic field sensor for precise localization of the robotic pill. FIG. 10A shows a scheme involving device location detection by a 3-D magnetic field sensor and an external magnet. FIG. 10B shows the sensing of magnetic field through a tissue at different lateral distances. FIG. 10C is a graph showing the measured 3D magnetic field at various distances from the magnetic source (dₓ=lateral distance [cm]; d_{y}=4.25 cm, d_{z}=1 cm; Magnetic source: 0.75 inch cubic Neodymium magnet). FIG. 10D shows a magnetic field through different materials including air, silicone, and tissue.

### DETAILED DESCRIPTION

The gastrointestinal (GI) tract harbors a diverse array of biomarkers, including proteins, extracellular vesicles, nucleic acids, and bacteria, which exhibit promising potential as disease markers. However, the extensive length of the GI tract (approximately 9 meters) and its challenging accessibility render conventional diagnostic procedures such as colonoscopies or endoscopies inadequate in reaching all regions of interest. Consequently, the inherent difficulty in monitoring relevant markers within the GI tract leads to incomplete, infrequent, or suboptimal data, limiting its clinical utility for medical decision-making. In response to this challenge, the development of smart capsules as liquid biopsy sampling platforms has emerged as a promising solution. Numerous sampling approaches utilizing liquid samples have been devised, primarily relying on passive diffusion. These methods involve the implementation of valves, mechanical actuators, and hydrogel absorbents. However, the collection of intestinal mucosal samples, which play a vital role in nutrient absorption and gut microbiome health, poses limitations. The viscoelastic nature and mechanical properties of the mucosa necessitate active sampling methods. Expanding the ability to analyze mucosal bioanalytes could significantly enhance our comprehension of gastrointestinal diseases and related pathologies.

Although other robotic pills have been used for various applications in imaging the GI tract with FDA approval, these FDA-approved products cannot collect samples. The disclosed pill uses a novel method designed to overcome this barrier. It can sample enteric mucus and capture neurochemicals, enteric extracellular vesicles (EVs), and bacterial nano-sized vesicles, also known as Outer Membrane Vesicles (OMVs), allowing easy and affordable repeat sampling of distinct GI tract regions of interest; this capability cannot currently be achieved through liquid biopsy, colonoscopy, or any extant micro-robotic systems. Such a tool has not been developed before. This is unique in exploring the GI Tract, as unlike image-based pills/pill cams that only capture images of the polyps or enteric cavity, this device can collect actual GI tract samples suitable for ultrasensitive and specific benchtop testing such as Omics analyses. This may integrate the ability to collect mucosa samples and autonomously sample the GI tract. It is contemplated that the pills capabilities also could be expanded to include to site-specific drug delivery and focal medical interventions. This integration may result in a medical device capable of mucosal sample collection, offering a comprehensive and minimally invasive solution for GI Tract exploration demands in an affordable and accessible manner. In contrast, the current state of the art devices to sample GI tract are designed for liquid sampling with a single location in mind.

Herein, a robotic pill design is presented specifically for sampling mucosa by employing a hydrodynamic screw mechanism which facilitates the collection of such a viscous sample. The robotic pill is designed to be easily swallowed and can be docked at a desired location using a magnetic docking mechanism. The pill's location within the gastrointestinal tract can be confirmed by a built-in 3D Hall sensor, and the hydrodynamic screw can be remotely activated once a target position is established. The rotational motion of the screw facilitates the collection of mucosal samples along the spiral path of the screw, directing the samples towards a dedicated collection chamber. While a Hall effect sensor is mentioned above, it is contemplated that three-dimensional volumetric spatiotemporal control could occur in any one of a number of ways, including external acoustic or magnetic fields or other types of affinity fields. Also, while a pill design is provided that is targeting mucus collection, based on the particular points of interest that the sample is not so limited to mucus, and other fluids and/or hard materials in the GI tract could also be collected with or without mucus. Nonetheless, where mucus is of primary interest, it is anticipated that this approach will offer a highly suitable method for minimally invasive sampling of the intestinal mucosa and mucus layer. Again, this capability cannot be achieved through either liquid biopsy nor colonoscopy currently and so this technology could enable easy, simple, affordable, and repeat sampling of distinct GI tract regions of interest, developing a robust platform for early disease diagnosis.

The device which hereinafter may be referred to as the “robotic pill” (but also may be referred to as the “G-I-ntellipill”, “Gintellipill”, or “S-PIRE” as an acronym for “Screw-based Pill for Intelligent Robotic Extraction”) includes, in the depicted exemplary form, an active component including a micro-screw conveyor driven by an electric motor for the purpose of rapid collection of viscous fluids as the main target, but also liquids, solids or a mixture of them.

The sampling/collection device can be of compact size cylindrical pill like shape for easy navigation in the GI tract, it includes a small window where the screw conveyor or hydrodynamic screw is exposed for contact with the sample to be acquired.

The collected sample can be transferred for storage and conservation/isolation to a collection chamber within the pill composed of one or several isolated compartments. The various compartments allow for multiple sampling at different times and/or from different locations along the GI tract. While the samples could be roughly isolated from one another by the various compartments utilizing a rotary multiple hermetic/isolated compartment chamber, sample cross-contamination reduction prevention also can be implemented by a screw reverse rotation cleaning system.

The location of the device can be accurately estimated in real time. For example, such location detection could be based on an algorithm model based on pH, transit time, motion and temperature, or magnetic location using a 3-axis Hall Effect magnetic sensor and an array of magnets; and/or a combination of the two methods for a more robust location system. In the exemplary form illustrated and for purposes of demonstration, location detection may be performed primarily using the 3-axis Hall Effect magnetic sensor. Additionally, external assisted location of the pill sampling device could be achieved by clinical/medical ultrasound imaging system.

Sample capture activation can be performed autonomously or on demand (remotely activated, user activated). For autonomous sampling, the pill microcontroller can be preprogrammed with the designated areas of the GI tract, sample time and/or pH where the system will activate the capture autonomously. In addition, remote on-demand user sampling activation is possible wirelessly by use of a computer or smart device. This is very convenient for precise sampling control by an operator/clinician when having an external imaging location unit like ultrasound, when using docking assist, or it is just desired to trigger a capture manually.

The robotic pill can also be retained/docked in a region of interest for prolonged periods of time by the action of a magnetic field for the purpose of prolonged or sequential sampling time in a specific area. This may be very convenient to hold the robotic pill for monitoring the absorbance of drugs, absorbance of nutrients in digestion, time evolution of mucus layers under certain conditions, and so forth. In addition, repositioning or positional adjustment of the robotic pill can also be possible by manipulation of an external magnetic field for fine correction of the position or for repositioning for enhanced sample capture, avoiding clogging, obstruction or accurate positioning.

The robotic pill is equipped with a wireless microcontroller which drive different sensors and actuators that allow the monitoring and storage of different variables of the system including pH, temperature, position, power consumption, time, motion, orientation, magnetic field, status, and so forth, which can be transmitted wirelessly to a computing or smart device for datalogging and future analysis of the system and correlation with the sample results.

Low power microcontroller with wireless Bluetooth or 400 MHz medical band transceiver can be used in the robotic pill device to drive the different sensors and actuators for optimized low power consumption. Communication with sensors and actuator can be done using the I2C, D/A, A/D, GPIO, SPI channels and ports, while the communication with external devices like computing or smart devices can be performed wirelessly.

Accordingly, three innovative tools can leveraged to detect, track, and molecularly characterize the targets within the GI tract. First, an integrated microcontroller system is developed to program sampling at a desired location. This sampling can be used to identify relevant neurochemical bioanalytes and spatially map their relative abundance, furthering the understanding of disease progression and health. Previous pill-based approaches are designed to sample at one time point and location, limiting the understanding of the marker concentration and distribution in different GI regions. Second, a hydrodynamic screw, driven by an electromechanical actuator, is utilized to achieve active collection of mucosal samples. After sampling, the captured markers can be isolated for further analytical evaluation. Past studies focused on imaging the GI tract via pills which do not sample the local microenvironment. Third, unlike past work that only focuses on the bacteria, unique components of the collected materials including extracellular vesicles (EVs), proteins and multiple neurochemicals used by the brain for physiological and cognitive processes can be examined (using for example ExoTIC or other such platforms such as described in U.S. Pat. No. 11,761,952). Thus, this technology platform can provide a precise, reproducible, and systematic method to collect and analyze markers, leading to improved disease monitoring, early-stage disease diagnosis and personalized medicine.

One aspect and aim of this disclosure is to provide and demonstrate a programmable robotic pill for sequential sampling. The design, fabrication, actuation, and function of the pill platform is demonstrated for sampling of mucosa and the control system to operate the untethered robotic pill. This involves developing and establishing a design for the robotic pill, including fabrication, sampling of mucosa though a hydrodynamic screw, and docking and positioning of the robotic pill. The pill can be equipped with a microcontroller embedded system for programmable sample collection. This system can allow for precise control over sampling location and timing. This provides an advanced approach to achieve reliable and reproducible collection of mucus samples.

Another aspect of this disclosure provides the platform for collection of gastrointestinal neurochemicals and extracellular vesicles, which is demonstrated herein in biomimetic phantoms and, for example, excised swine intestine, but more generally could be applicable to any gastrointestinal tract. A biomimetic model can be used to perform sampling at a target site that will result in the capture of a sufficient quantity of gastrointestinal bioanalytes to provide and demonstrate a reliable diagnostic result. The collected EVs can be tested via mass spectrometry for predictive markers for disease. Combining the high surface collection area and prolonged sampling times should result in a higher collection of bioanalytes than current approaches (no retention and one-point sampling). In addition, the selective collection can be evaluated using fluid flow to simulate the challenges of sample collection in dynamic environments.

Again and herein, an intelligent robotic pill device is disclosed for active low/non-invasive localized untethered collection of samples in the GI tract. The exemplary robotic pill is designed to collect viscous fluids, specifically the gastrointestinal mucosa layer, but it is also capable of collecting liquid, small solids, or a combination of them from the GI tract. The capsule can be introduced in the GI tract of a human or animal by swallowing or a surgical opening and recovered by excretion. The capsule collects the sample autonomously on the preprogrammed zones of the GI tract or wirelessly remotely activated on the designated areas. The capture of mucus or biofluid is done by an active motorized micro screw conveyor and stored in an isolated/hermetic chamber. The recovery of the pill device is done by excretion and the pill device cleaned for further recovery and analysis of the sample. Sampling of GI mucosa is of special interest as the GI mucosa contains bioanalytes including proteins, extracellular vesicles, nucleic acid, bacteria, neurotransmitters, and other biomarkers. Continuous collection and analysis of these markers can enable detection and following of unhealthy tract and gut related diseases at early stages such as irritable bowel syndrome, intestinal dysbiosis, gut neurodevelopmental disorders and cancer.

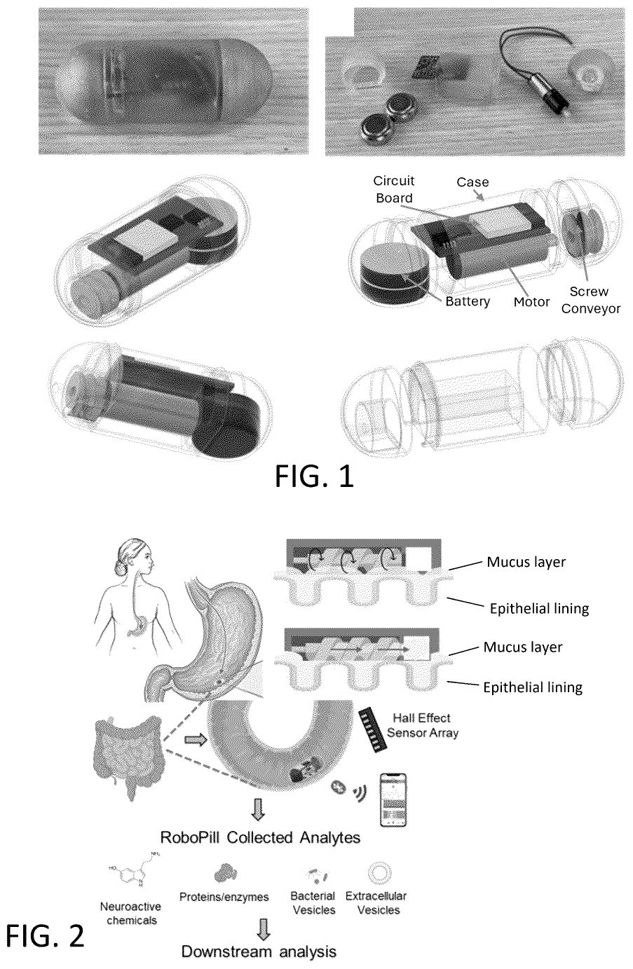

With reference being made to FIG. 1, an exemplary robotic pill incorporates a multiple component including: a hydrodynamic screw actuator, magnetic docking system, a control chip, a 3D-printed polymeric chassis, miniature sensors, and communication antenna all compactly integrated within the pill. In this particular exemplary form and by integrating a hydrodynamic screw mechanism, a magnetic actuation and docking system, and a wireless microcontroller system equipped with 3-axis Hall effect magnetic sensor and Bluetooth communication antenna in one pill, a combination is provided that is specifically designed for a site-specific and more effective sampling of viscous samples. The rotational motion of the screw enables active sampling of viscous samples along its spiral path, directing them toward a dedicated collection chamber. The robotic pill comes in a swallowable size (l: 4.3 cm, h: 1.6 cm) and can be retained at a targeted location (for example, at a particular location along the GI tract) using the magnetic docking mechanism embedded within the pill as will be described below. Once the robotic pill's location is confirmed via the integrated 3-axis Hall effect sensor, then the hydrodynamic screw can be activated remotely through the wireless Bluetooth communication (or other wireless communication) in the microcontroller system, ensuring site-specific sampling. After controlled magnetic navigation of the robotic pill to a designated collection site, at this site the robotic pill collects and securely stores a viscous sample within a modular, detachable, and disposable chamber. This collected sample can then subsequently analyzed (for example, for the presence of protein bioanalytes such as for example hemoglobin). Such a design provides a minimally invasive approach that enables more efficient, targeted, and on-demand sampling of viscous samples.

Returning to the specific structure of the exemplary robotic pill, the exemplary robotic pill integrates several functional components including a 3D-printed hydrodynamic screw actuator, magnetic docking system, control chip, miniature sensors, and communication antenna-all compactly housed within a 3D-printed polymeric chassis. The hydrodynamic screw, powered by an electromechanical motor, can rotate up to 600 rpm without any load. Neodymium magnet encapsulated within the pill structure allowed for external magnetic field manipulation, ensuring the pill remained in the desired area for an extended period. The polymeric chassis securely holds each component in place.

The active sampling device or robotic pill comprises a micro screw conveyor driven by an electric motor for rapid capture and transport of the biological sample to the embedded collection chamber. The system includes a battery-operated circuit board with a microcontroller for remote or autonomous activation of sample capture in the designated regions of the GI tract. In addition, the circuit board includes power drivers for the motor and different sensors for location capabilities. The robotic device shell/enclosure is of compact size with a cylindrical pill-like shape allowing for easy navigation inside the GI tract. The robotic device shell/enclosure also has a flat side along the large axis with a small rectangular window or opening to expose the micro screw conveyor to the sample.

In one particular form, a FormLabs 3D printer can be used to create the pill structure including storage chamber with three selectable collection compartments, a hydrodynamic screw for mucosal sample collection, support for an electrical rotational micromotor, and cavities to accommodate permanent magnets for positioning.

The hydrodynamic screw may be 3D-printed and attached to an electromechanical motor to enable rotation of the screw. In the exemplary form illustrated, the robotic pill was constructed from multiple integrated components, including a solid polymeric chassis and a hydrodynamic screw, both fabricated using a Form 3+ 3D printer (Formlabs).

As depicted, the exemplary also design incorporated a 9.5×4.5×1.5 mm rectangular neodymium magnet (K&J Magnetics), an electromechanical actuator, two batteries, and a microcontroller with integrated sensors and an antenna. The two magnets are encapsulated within the pill structure enabling manipulation of the pill remotely and external to a body. This magnetic retention ensures that the pill remains in the desired area for an extended period. The polymeric chassis holds everything in place. The pill's dimensions (length, diameter, and thickness) can be designed to meet FDA-approved ingestible device standards: 16 mm length, 6 mm diameter, and 1.5 mm thickness.

The control electronic board was designed in a compact format (11.4×21.3 mm²) to optimize low power consumption. A featured low-energy RF wireless microcontroller (STM32WB5MM, STMicroelectronics) was selected for its small size, ultra-low power consumption, integrated wireless communication through a dedicated M0+ processor with embedded antenna, 32-bit Arm Cortex M4 CPU processor capacity, and available ports for peripherals connectivity. The board also integrated a 3-axis (3D) Hall sensor (ALS31300, Allegro Microsystems), an accelerometer/gyroscope (ISM330, STMicroelectronics), an instrumentation amplifier (INA333, Texas Instruments), and a low-dropout (LDO) regulator (LP5907, Texas Instruments). The instrumentation amplifier was incorporated for future sensor integration. It is contemplated that, in some forms, there could be a pH sensor for advanced positioning and GI tract recognition The motor control was managed by a power driver block (DRV8833, Texas Instruments), driving an ultra-small 6×14 mm² 3V DC geared micromotor with a torque of 25 g-cm to actuate the screw. The control electronic board broadcasted up to 6 dBm of power at 2.4 GHz, enabling wireless communication to receive commands and status data including timing, 3D position, GI position, and battery level. The system (electronic board, signal transmission, and motor) was powered by two 1.5V lithium batteries, approved for medical use, to meet the power demands of the actuators. Alternative biobatteries that harvest energy from the GI tract, improving biosafety, may also potentially be employed. The PC board was designed using Autodesk Eagle PCB software, and microcontroller firmware was programmed in C language with STM32Cube design tools.

To comply with FDA biocompatibility recommendations for medical devices contacting the human body, the pill may be fabricated with materials such as PMMA that are FDA preapproved for use without requiring further biocompatibility testing. The electronics and battery can be hermetically enclosed and coated with parylene (FDA approved) to prevent leeching following FDA recommendation.

For the sake of comparison, Table 1 below provides a comparison of a microfluidic sampler versus this newly-disclosed robotic pill.

TABLE 1

| Novel Robotic Pill | Microfluidic Sampler |
|---|---|
| Active capture of sample (motorized) | Passive capture (absorption) |
| Sampling time (10-100 seconds) | Sampling time (take hours) |
| Larger sample volume capacity (>500 μL) | Limited sample volume |
| | collection (<20 μL) |
| Isolated chamber (contamination | Exposed chamber (open to |
| reduction) | contamination) |
| Modular removable multiple isolated | Multiple exposed chambers |
| compartment chamber | |
| Able to capture high viscosity, small solids, | Low viscosity and liquid |
| liquid sample and combination of them | sample only |
| Wireless communication and activation on | Not available |
| demand (Bluetooth or 400 MHz medical | |
| band) | |
| Electronic programmable capture activation | Programmable sampling |
| in different regions, times, or by pH | only by degradation of |
| | enteric coating by pH |
| Able to datalog/store/record different | Not available |
| variables including time, pH, temperature, | |
| collection times, motion, power | |
| consumption/battery life | |
| Magnetic docking/motion control | ✓ |
| On pill position estimation | Not available |

From Table 1 above, it can be observed that, due to the viscous/mechanical properties of mucus, it is very difficult for mucus to be captured by passive methods including one-way valves, osmotic gradients or absorption of from earlier microfluidic sampling devices.

One of the most unique elements of the sampling device is the micro screw conveyor or hydrodynamic screw, which is an active element designed to capture viscous samples in hard-to-reach regions, such as the GI tract. Given the viscous properties of mucus (e.g., intestinal mucus layer), passive methods including one-way valves, osmotic gradients, or absorption are ineffective for sample capture of such viscous materials. Screw conveyors, however, are well-suited for this application and are herein adapted for active sampling and capture of viscous samples using a small-factor screw element. The screw conveyor includes a helical bladed screw enclosed within a barrel inside a housing and with an opening or small aperture exposed or submerged on the fluid or material to be extracted. This opening or small aperture on the housing permitting passage to the barrel, allows samples to interact with the screw's surface, where they are adhered and transported. Rotation of the bladed screw pushes or transport the materials in the axial direction of the screw. Flow or transported volume is a function of the helix diameter, distance between helixes, exposed area, immersion height and mechanical properties of the sample. This principle is adapted for a small factor capture and transfer of viscous materials as depicted in top right panels of FIG. 2. When the micro screw conveyor is in contact with the viscous fluid, the viscous fluid adheres to the surface of the micro screw conveyor, then the viscous fluid is transported axially by rotation of the screw and transferred to the chamber compartment where the sample is stored for later analysis. Transport flow is a function of helix diameter, separation between helixes, angular rotor speed and thickness of the sample. As the screw rotates, it generates a force F_{d} that drags the sample axially towards the storage compartment, as visualized in the upper rightmost panels of FIG. 2. The drag volumetric flow rate Q_{d} of the sample depends on several design parameters, including barrel diameter D, screw length L, thread channel width H, depth d, angular speed ω, and helix angle θ, as shown in the equation below:

$${Q}_{d} = \frac{D \cdot ω \cdot d \cdot H}{2} cos θ$$

The angular speed ω is directly influenced by the sample viscosity μ, assuming a constant force delivered by the motor. This, in turn, affects the resulting volumetric drag flow of the sample Q_{d}. As the fluid viscosity u increases, the angular speed w and the drag volumetric flow rate Q_{d} will be reduced due to greater resistance to fluid transport. In modelling and testing the transport of viscous materials it was observed that higher viscosity necessitates a greater drag force, which, under a constant motor-generated force, reduces the capture rates as viscosity increases. Conversely, lowering the viscosity of the material of interest, enhances the capture rate. Accordingly, over a fixed duration, the capture volume decreases with increasing viscosity due to the greater resistance to fluid transport. For purposes of proof of concept, early prototypes were able to capture 100 μL of viscous fluids within 15 seconds with a storage capacity of more than 500 μL.

It is contemplated that the screw performance could be further evaluated through in vitro experiments using phantom tissue (ultrasound gel) as a model of the intestinal mucosal layer, varying thickness and viscosity from 40 to 160 Kcps or Pa-s and quantifying captured mucosa. To assess the pill's ability to collect tracer particles from distinct locations and check for cross-contamination, an intestinal model could be created with three distinct regions containing different 200 nm fluorescent nanoparticles (nile red, sky blue, and yellow). The samples captured could be inspected in each of the collection compartments to verify the screw's ability to perform sequential spatial sampling at different locations. The screw may have contamination prevention features such as reverse rotation for system cleansing and a selectable rotary collection chamber with three isolated compartments. Additionally, in some modes of operation, the pill could be re-oriented to ensure perpendicular positioning of the screw against the intestinal lining, including re-orienting using an external magnetic field to align an embedded magnet. Applying feedback-controlled torque to the entire pill structure will reorient it according to the magnetic field lines from the external actuator. In addition, the current of the motor driving the helical screw could be monitored under different conditions of capturing mucosa described above to model and detect when the pill is capturing samples.

Sampling duration and volume of collected samples (mL) might be further investigated to account for sample viscosity, screw geometry, rotation speed, and blade angle and separation, as well as modelling pill's expected human/animal GIT location and sequential capture timing to reach the desired GI tract zone.

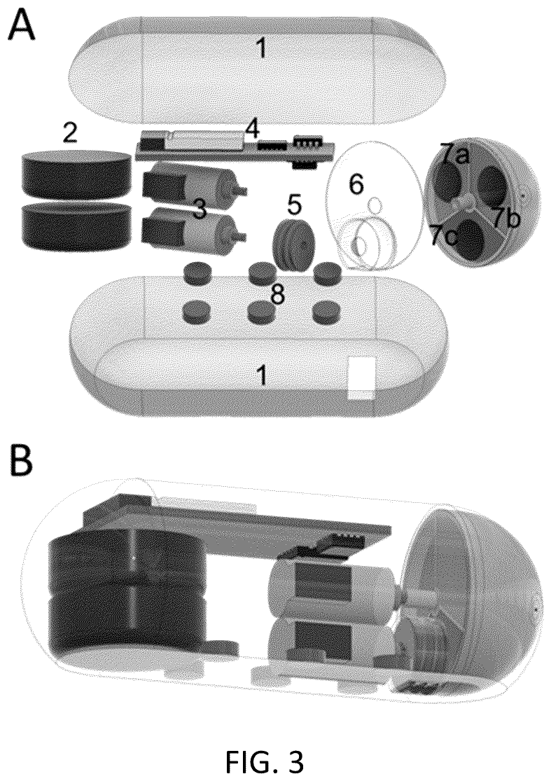

As depicted in FIG. 3, additional elements include a chamber with multiple compartments for sequential sampling in different times or sections of the GI tract, and an array of micro magnets for magnetic docking and motion control.

Capture/storage of the sample from multiple points (that is different sections and/or different times) may be performed using a stepper motor driven rotary chamber equipped with multiple isolated compartments (see e.g., items 7a, 7b, and 7c) allowing for cross contamination reduction. In addition, a screw reverse rotation cleaning system may be implemented to remove any old sample from the conveyor before the collection of new samples to minimize contamination. Additionally, an enteric coating can be applied to the pill body as an extra protection layer to be dissolver in the designed environment helping to reduce device contamination.

The collection chamber is designed as a modular element that can be detached and replaced, allowing for easy access to the captured sample. The hydrodynamic screw is embedded in the chamber for single use to reduce contamination. In addition, the chamber can be fabricated for different volume capacities, screw designs (even different tools) for custom specific needs or applications including biopsy extraction by using the same capsule core and a sharp cutting screw or tool, giving versatility to the pill platform.

Magnetic docking and motion control of the pill can be done with the addition of an array of micro magnets in the pill structure and the action of a remote/external magnet.

As an objective was to develop and evaluate a magnetically actuated robotic pill designed for site-specific, active sampling of viscous fluids, various aspects of its operation were further investigated and characterized which are now described. Some investigated components and aspects included the integration of a magnetic actuation system for precise motion control and positioning, a wireless microcontroller equipped with 3-axis Hall effect magnetic sensor and Bluetooth Low Energy (BLE) communication antenna for remote operation, and a modular, detachable collection chamber for secure sample storage. The performance of the magnetic actuation and BLE communication systems were tested under tissue-mimicking conditions to evaluate docking capacity, positioning accuracy, and power signal integrity. In vitro experiments were conducted using mock shear-thinning viscous samples of varying viscosities to quantify collection efficiency, with biological analytes such as hemoglobin and bovine serum albumin incorporated to simulate broad applicability in real-life sampling conditions.

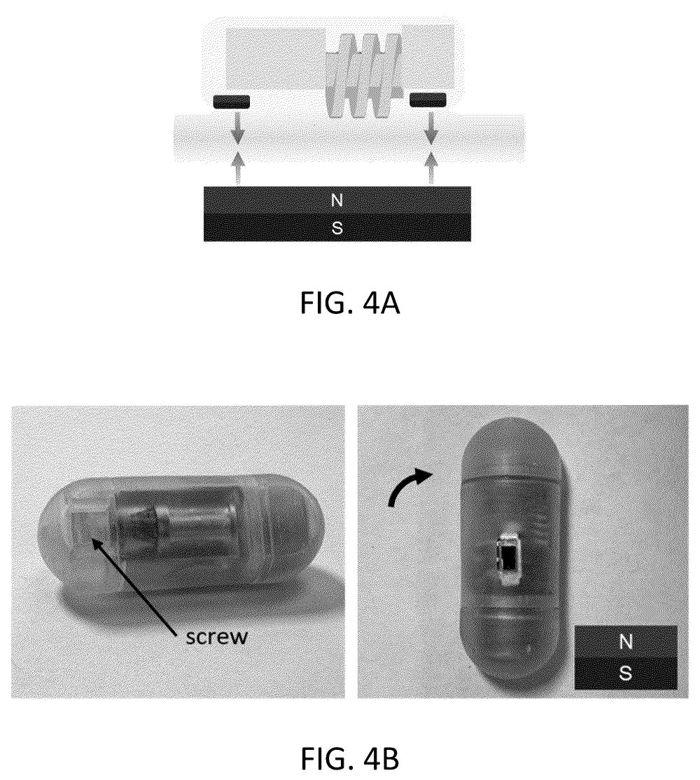

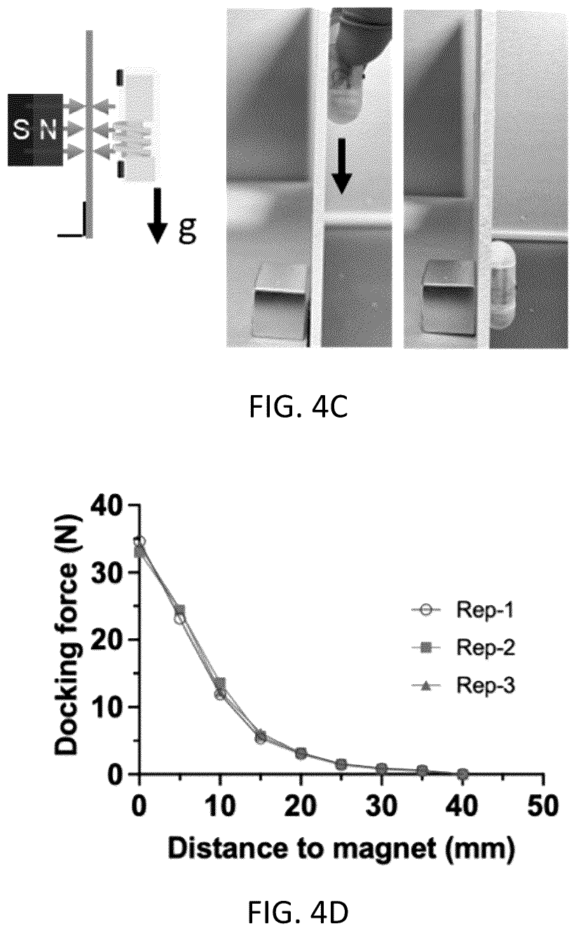

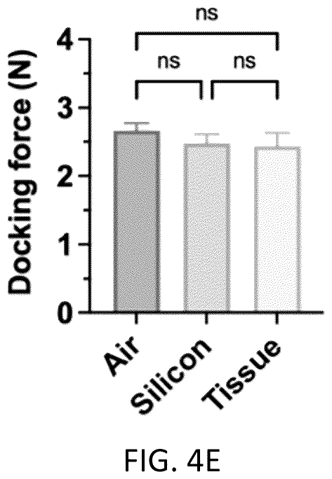

Docking or holding the position of the sampling device inside the GI tract helps ensure accurate positioning in the designated region of the GI tract. Although, due to the active capture mechanism, the sampling times are short (˜100 μL within 15 sec), applications where timed collection of samples in the same zone at different times may be required. With reference being had to FIGS. 4A-4E, docking can be achieved by retaining the pill in the desired position by magnetic attraction between the pill micromagnets and the external magnet.

The robotic pill may be able to be retained or docked in a target region using a magnetic field, allowing for a controlled sampling duration in a specific location. Incorporating ferromagnetic material or permanent micro-magnet within the robotic pill enabled the robotic pill to be attracted by external magnet. This attractive force depends on factors such as shape, magnetization, media permeability, and is inversely proportional to the square of the distance between the magnets. Retention of the pill against gravity was demonstrated using an external neodymium magnet as depicted in FIG. 4C.

The magnetic attraction generated docking forces exceeding 2.5 N at a 2.5-cm separation, which is over two orders of magnitude higher than the 0.098 N required to hold a 10-g pill against gravity and well above the 0.9 N of radial peristaltic forces of the intestine.

Given that magnetic attraction decreased with increased distance from the magnetic source as depicted in FIG. 4D, this factor must be carefully considered in the design of the actual docking system for biomedical applications. With reference being made to FIG. 4E, testing demonstrated consistent retention forces of approximately 2.5 N across various materials including silicone and tissue (of 25-mm thickness), with air as a control. This consistency is attributed to the similar relative magnetic permeability of those non-magnetic materials to that of air (Hr=1). To arrive at these values, the force exerted by the external magnetic field was measured by attaching the pill to a 500 N digital force gauge (Mxmoonfree) and moving a 25-mm cubic neodymium magnet (K&J Magnetics) toward the pill at various distances and angles. To assess force over different barrier materials, such as polydimethylsiloxane (PDMS, Sylgard) and pork belly tissue (purchased from a local supermarket), each with a thickness of 25 mm, were placed between the pill and the external magnet.

These results suggest the potential of docking the robotic pill in specific regions of the body, such as the gastrointestinal tract, to enable prolonged sampling and facilitate time-dependent or sequential acquisition. Such functionality holds potential for targeted studies on the absorption of drugs and nutrients, as well as the time-dependent behavior of mucus, which could aid in disease and treatment evaluation.

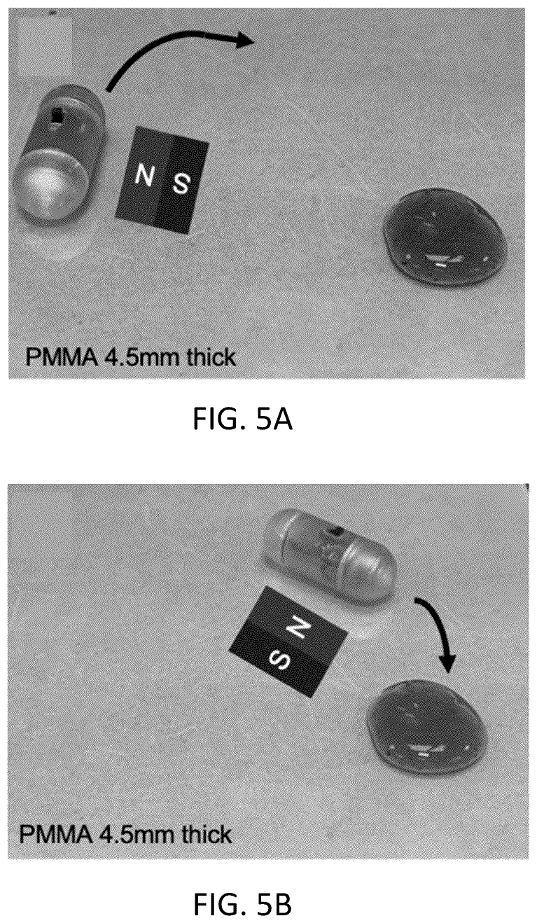

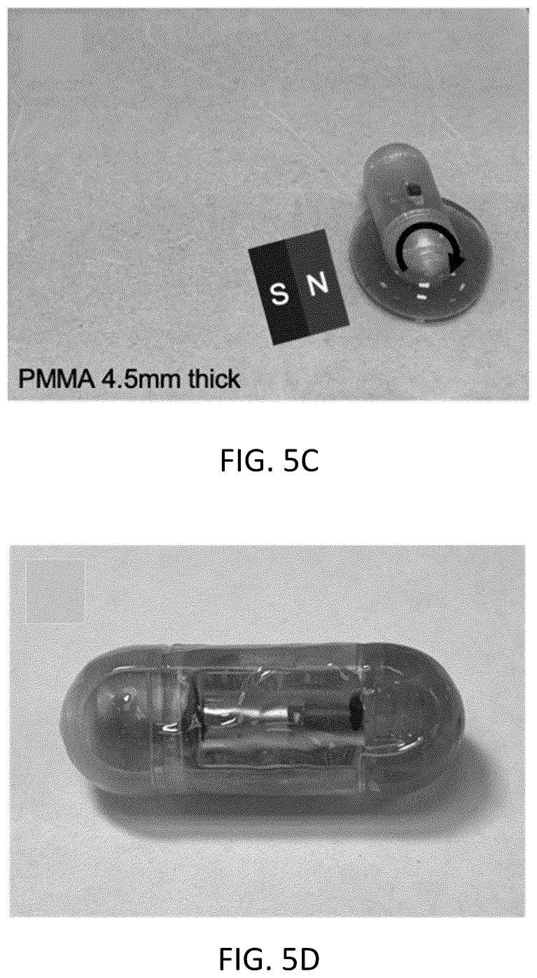

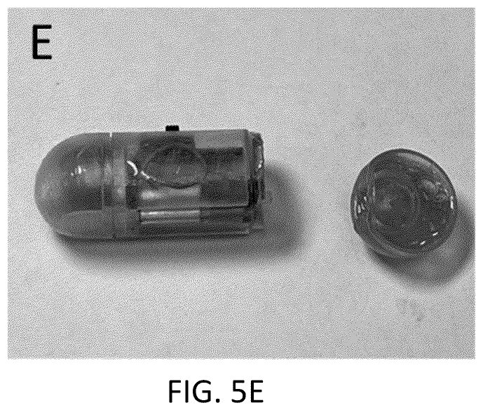

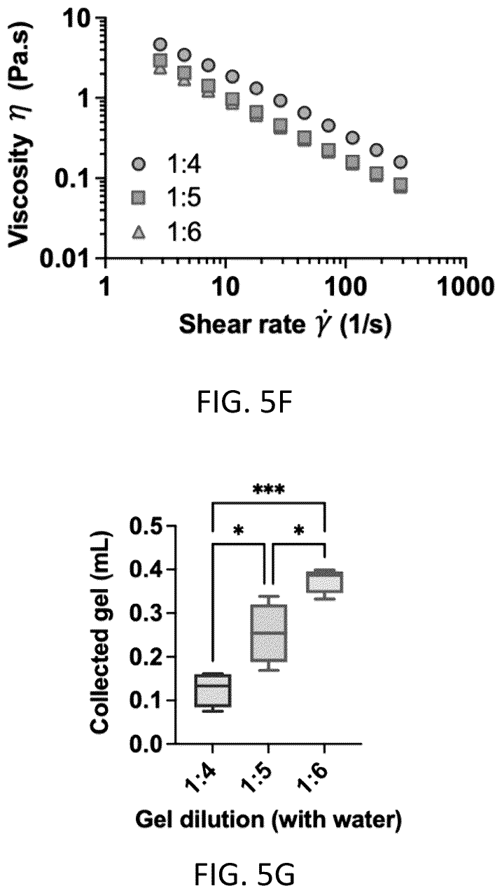

Motion control of the robotic pill can be achieved by magnetic attraction and interaction of the micro magnets in the robotic pill and an external magnetic field produced by a larger external magnet utilizing a magnetic actuation system. The larger external magnet exerted both torque and magnetic force, acting on the micro-magnet embedded within the robotic pill. This interaction aligns the micro-magnet's magnetic moments with the magnetic flux lines of the external larger magnet, enabling controlled movement and positioning of the pill within the target region. The attractive pulling force is proportional to the micro-magnet's magnetic moment and the gradient of the larger magnet's magnetic flux density. As the external magnet moves, the robotic pill aligns and adjusts its orientation and position accordingly through magnetic alignment and attraction. As depicted in FIGS. 5A-5C, which shows such motion control in an exemplary fashion, this guided motion effectively directed the robotic pill to the designated sampling region, ensuring precise positioning for sample collection. This function is very useful when it is desired to move the pill to a specific location. In addition, it can also improve the sample captured volume by applying a small force of the pill towards the surface where the pill is capturing sample to allow better interaction with the sample.

Once the robotic pill is positioned in the target sampling region, the screw conveyor mechanism may be activated, enabling the capture and secure storage of viscous sample into a modular, detachable, and disposable chamber which chamber is depicted in FIGS. 5D and 5E. The volume is determined by the size of the robotic pill's collection chamber; hence this modular design could facilitate easy interchangeability, allowing different chambers of varying volume capacities to be used based on specific sampling requirements (for example, the illustrated design has a maximum volume capacity of 500 μL).

The robotic pill was evaluated for its ability to capture viscous materials, such as ultrasound gel, at various dilutions in water. Hydrogel (Aquasonics clear ultrasound gel 03-08, Parker) was diluted in water with different dilution factors, such as 1:4, 1:5, and 1:6 (v:v). Both mucus and the diluted ultrasound gel are non-Newtonian fluids with shear-thinning property, which means that material viscosity decreases with increasing shear rate. Viscosity was measured using Discovery Hybrid Rheometer (Discovery HR-3, TA Instruments). The diluted gel was run under flow sweep test with shear rates applied ranging from 2-300 s⁻¹. The viscosity was determined based on the shear rate. The diluted gel in the experiment exhibited shear-thinning property where viscosities decreased with increased shear rates as depicted in FIG. 5F. This behavior aligns with trends observed in mucus, as the slope of the log-log plot in the experiments ranged from −0.74 to −0.78, closely matching the reported mucus slope of −0.85. Moreover, at room temperature and a shear rate of 11 s⁻¹, a representative shear rate comparable to the frequency of airway cilia beating for mucus clearance (12-15 s⁻¹), a gel dilution of 1:6 (v:v) exhibited a viscosity of 0.87 Pa-s, while 1:5 and 1:4 dilutions exhibited viscosities of 0.97 and 1.86 Pa-s, respectively. According to the literature, physiological mucus from stomach, small intestine, and colon, exhibits viscosities ranging from 0.06 to 1.78 Pa-s at shear rate of 10 s⁻¹. Hence, the gel dilutions used in this work closely resemble the viscosity range of physiological mucus.

The ability to collect mock viscous samples using hydrogel dilutions at 1:4, 1:5, and 1:6 (v:v) was tested, each exhibiting different viscosities as described above. The sampling process was conducted over a 60-second period, during which the robotic pill actively collected the gel into its detachable chamber. After collection, the chamber was removed, and the collected samples were weighed to determine the captured volume. The density of the hydrogel dilutions was assumed to be approximately 1 g mL⁻¹, equivalent to that of water, allowing direct conversion from mass to volume.

The results of those tests are illustrated in FIG. 5G. In FIG. 5G, it can be seen that, within 60 seconds, the robotic pill successfully collected approximately 375 μL of the 1:6 (v:v) gel dilution. However, as viscosity increased, the captured volume decreased to about 250 μL for the 1:5 (v:v) dilution and around 150 μL for the 1:4 (v:v) dilution. This volume reduction underscores the relationship between fluid viscosity and drag force Fa, where the force required to transport the sample increases with viscosity. Under a constant driving force from the motorized screw, the flow rate Q_{d} decreases with increasing viscosity due to greater resistance to fluid transport. Notably, without activating the screw conveyor mechanism, the exemplary robotic pill was unable to collect the viscous sample. Additionally, based on calculations, increasing the collection time is expected to increase the total captured volume, assuming a steady-state capture rate for a given viscosity. For example, a gel dilution of 1:6 (v:v) demonstrated an experimental capture rate of ˜6.25 μL s⁻¹, requiring approximately 80 seconds to fill up a 500 μL chamber. In contrast, a gel dilution of 1:4 (v:v) showed a reduced experimental capture rate of ˜2.5 μL s⁻¹, taking around 200 seconds to reach the same chamber's maximum capacity of 500 μL.

Therefore, the results demonstrate robotic pill's capability to actively collect highly viscous fluids that are otherwise challenging to obtain through passive diffusion methods. Moreover, the rapid sampling speed, facilitated by the motorized hydrodynamic screw mechanism, suggests the potential of such a robotic pill to enable future localized capture of viscous samples in specific anatomic regions, such as the GI tract.

The sampling capsule device is mainly designed for capture, separation, and storage of biological sample and/or biomarkers in the GI tract for further post-processing/analysis. Accordingly, the robotic pill was also evaluated for its capability to collect biological samples such as hemoglobin (Hb) from a simulated mock mucus layer (e.g., diluted ultrasound gel). Both mucus and the diluted ultrasound gel are non-Newtonian fluids with shear-thinning property, which, with reference to FIG. 5F, means that the material viscosity decreases with increasing shear rate.

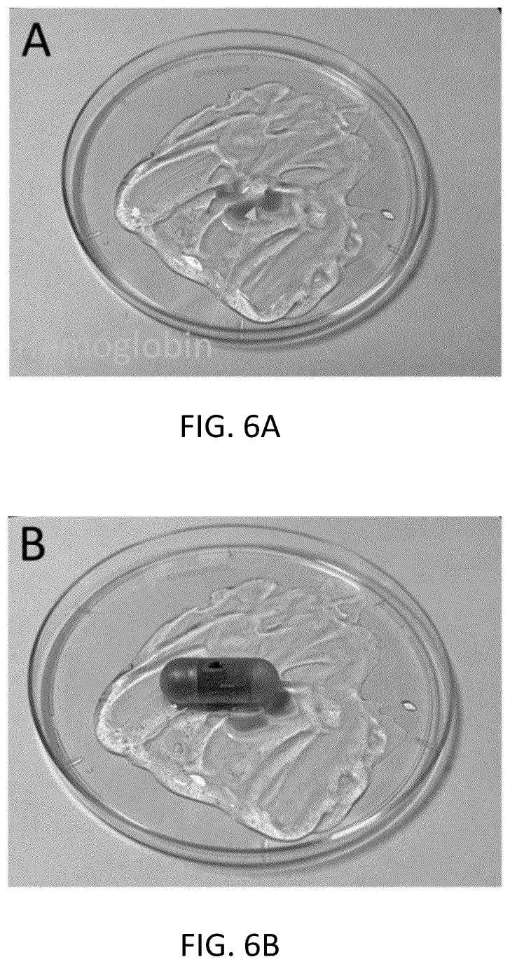

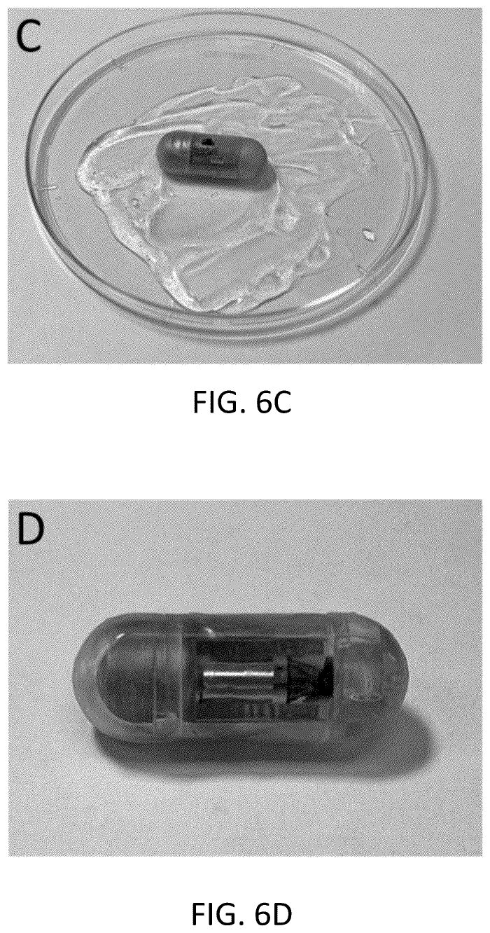

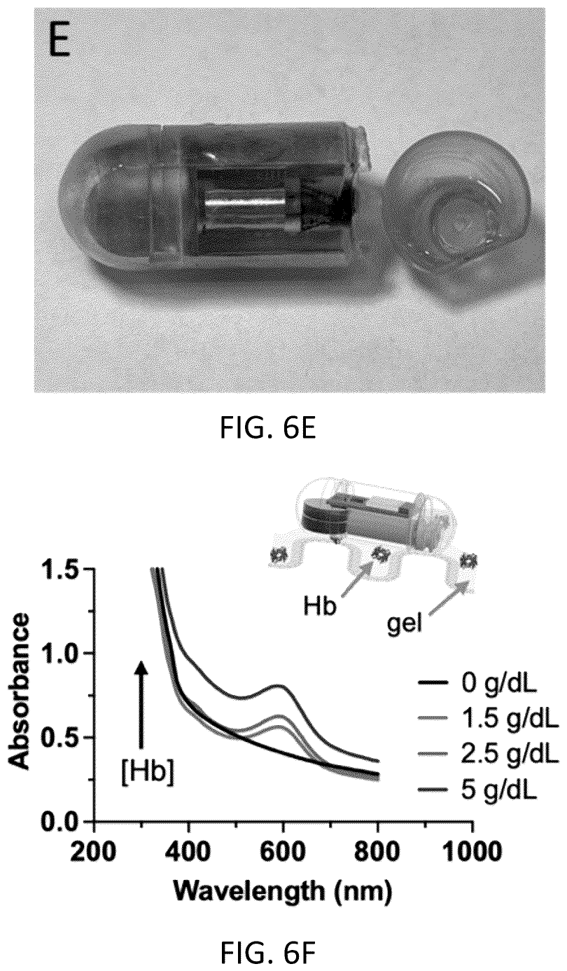

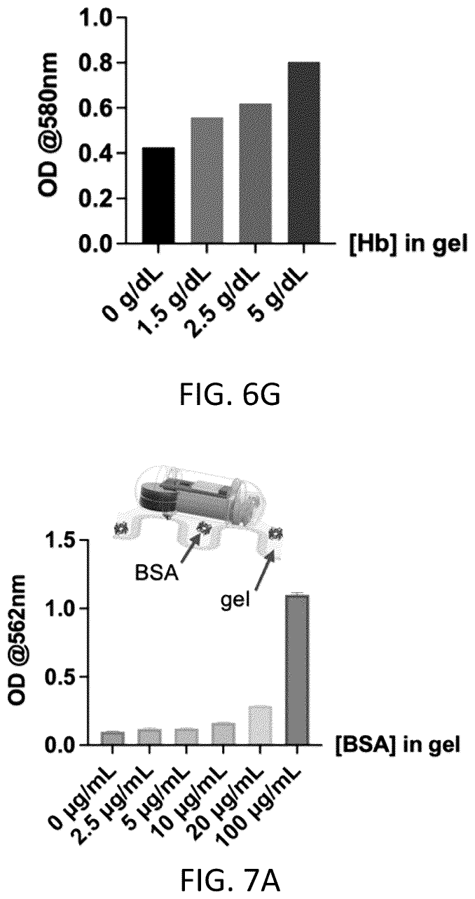

FIGS. 6A-6E depict the recreation of a bleeding on viscous mucosa, using gel as the viscous media, and further demonstrate the rapid active capture and storage of the biomarker by the robotic device for further ex-situ analysis. First, Hb solutions were prepared at various concentrations (0, 3, 5, 10 g dL⁻¹) by dissolving Hb powder (Sigma-Aldrich, H7379-5G) using PBS as the solvent. Each Hb solution was mixed with an equal volume of 1:5 (v/v) hydrogel, resulting in a final Hb concentration that was half of the initial preparation. Then, in FIGS. 6A-6C, the robotic pill was directed to a capture location using magnetic guidance, where it successfully actuated and docked to collect the viscous sample containing Hb, visualized by red Hb dispersed in it. The collection process was conducted over a period of 60 seconds to ensure optimal sampling, based on our theoretical calculation (assuming a continuous fluid supply). Even with the most viscous 1:4 (v:v) diluted gel sample, a 60 second duration should be sufficient to fill up the entire collection chamber volume. Regardless, it should be noted that this experiment aimed not to extract all the sample but rather to detect the presence of Hb that might be a potential application to identify signs of “bleeding”, so complete collection would likely still be acceptable. Following collection, the head of the robotic pill (i.e., the collection chamber) was detached to facilitate the retrieval of the viscous sample. The hemoglobin (Hb) concentration in the viscous samples was measured using a Guaiac test. Gum guaiac (Santa Cruz Biotechnology, sc-215116A) was dissolved in 200-proof ethanol containing 3% hydrogen peroxide to prepare a 67% (w/v) Guaiac solution, which was subsequently added to the previously collected viscous samples containing Hb. Based on scanning the spectra, the peak optical density at 580 nm (OD₅₈₀) was recorded for each sample. The results demonstrated a linear increase in absorbance with higher Hb levels as illustrated in FIGS. 6F and 6G.

Thus, it was demonstrated that the robotic pill could collect a Hb-containing mock viscous sample for detection of different Hb concentrations inside the sample. This capability could pave the way for future applications in quantifying intestinal bleeding from epithelial linings, which is a significant parameter to monitor in inflammatory bowel diseases, such as Crohn's disease, as well as some cancers.

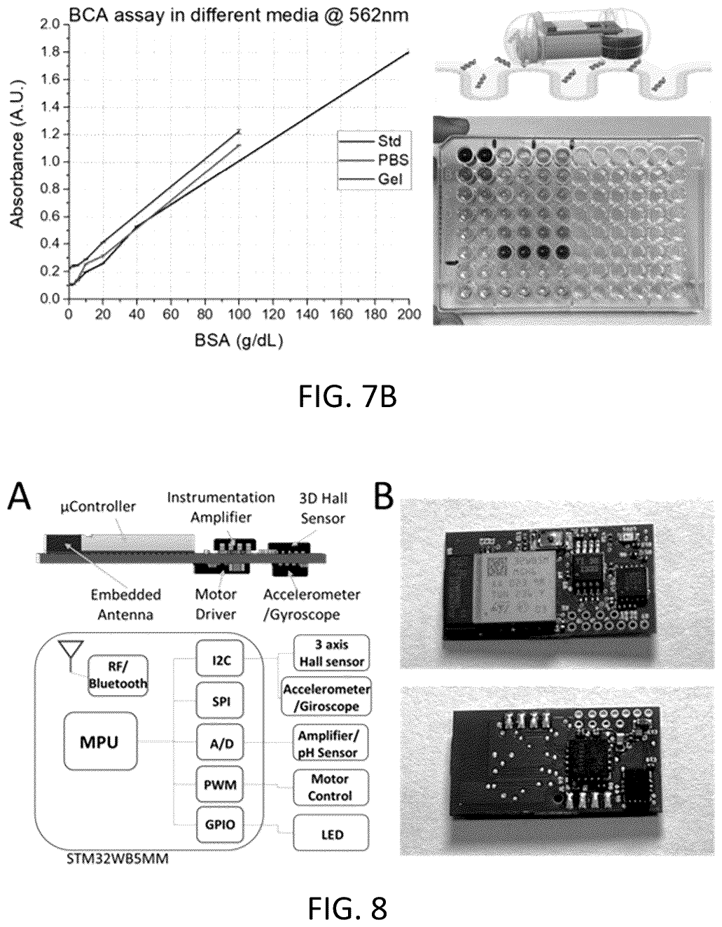

Another example is the capture of proteins like bovine serum albumin (BSA) in the viscous matrix by the robotic device for subsequent external quantification with colorimetric Pierce microBCA assays using spectrophotometry, which is depicted in FIGS. 7A and 7B. BSA solutions were prepared at various concentrations (0, 5, 10, 20, 40, and 200 μg mL⁻¹) using PBS as the solvent. Each BSA solution was mixed with an equal volume of 1:5 (v/v) hydrogel, resulting in a final BSA concentration that was half of the initial preparation. Similar to the Hb collection, the robotic pill was employed to collect the viscous samples containing BSA. The collection process was conducted over a period of 60 seconds to ensure filling of the collection chamber. Following collection, the collection chamber was detached to facilitate the collection of the viscous sample. The concentration of BSA in the gel samples was determined using a microBCA kit (Thermo Scientific, 23235). The optical density at 562 nm (OD₅₆₂) was recorded for each sample. As is perhaps better illustrated in FIG. 7B, it was found that the absorbance increased linearly with BSA concentrations. This result indicates the robotic pill's broader potential for collecting and analyzing diverse biological samples. These findings hold promise for viscous sample collection for future biomarker analysis of GI tract layer, particularly with implications for minimally invasive medical diagnostics and research, contributing to the ongoing development of innovative technologies in this field.

On-pill control electronics is designed for small form factor, ultra-low energy consumption and wireless operation of the sampling device. As illustrated in FIG. 8, the on-pill control electronics comprise a wireless RF low energy microcontroller, motor driver, 3D Hall magnetic sensor, accelerometer/gyroscope, low signal amplifier for pH sensor reading, voltage regulator and battery.

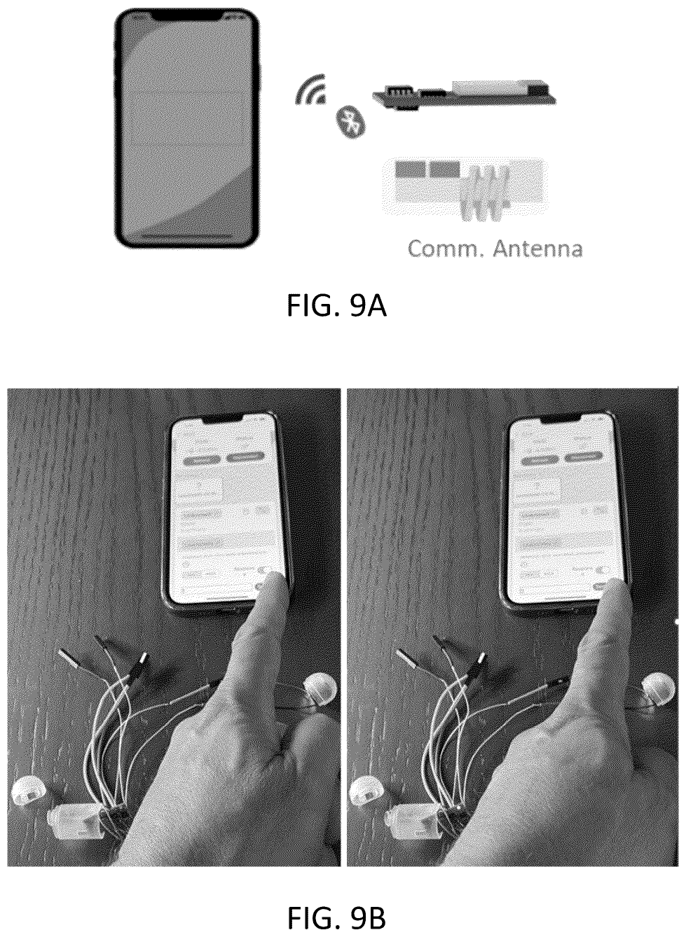

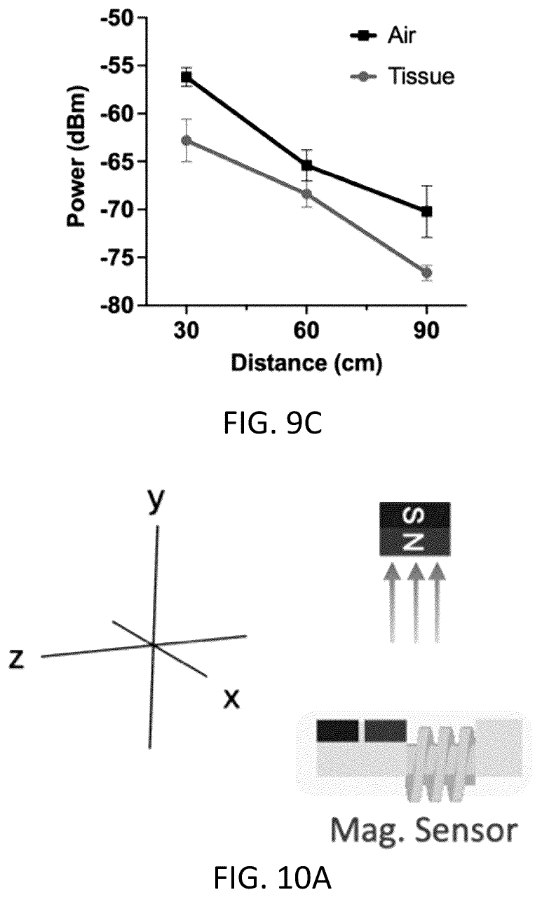

The control electronics board uses a low energy RF wireless microcontroller in the Medical Implantable Communications Service MICS 400 MHz band or Bluetooth low energy 2.4 MHz band. Such a low power wireless microcontroller (e.g., STM32WB5MM) can communicate and control the onboard peripherals through the I2C, SPI, A/D, PWM and GPIO ports, along with storing and processing the acquired signals to obtain pH, magnetic field, position, acceleration, battery life, estimated location, drive the motors and sense their current, activate sampling and timing. In addition, it communicates with external devices including, smartphones, tablets and computers or custom device to transmit the variables for data logging, supervision and/or remote operation of the sampling device. FIGS. 9A-9C demonstrate the capability of the robotic pill to be controlled wirelessly via Bluetooth using a smart phone. The capabilities of the microcontroller (1 MB+256 kB memory) allow for temporary data storage of the acquired variables for wireless transmission and data logging by an external device. The embedded Arm Cortex M4 processor can be capable of implementing artificial intelligence/machine learning for autonomous operation of the robotic pill device. In addition, power drivers were incorporated to control motor actuators, while 3-axis Hall effect magnetic sensor and an accelerometer/gyroscope to track precise localization, motion, and orientation

As depicted in FIGS. 9A and 9B, an electronic feature of the system can be that the wireless Bluetooth communication (or other wireless communication modality) allows a smart device to control and monitor the robotic pill by accessing data from its embedded sensors and variables in the control board, including the magnetic sensor, gyroscope, amplifiers, power drivers, status, and more. Additionally, through this wireless Bluetooth communication (or other communication link), sample capture by the hydrodynamic screw mechanism could be activated or deactivated via a smart device. This functionality facilitates precise sample collection in targeted regions of the body, with potential for future application under the control of an advanced user or a qualified technician/physician, enhancing clinical usability.

Further, the signal strength and integrity were evaluated by enclosing the robotic pill in a 25-mm thick pork belly tissue, reproducing a GI tract barrier condition. Again, to manage wireless operations, the pill uses the dedicated ARM Cortex M0+ CPU of the STM32WB5MM microcontroller, as described above. Bluetooth Low Energy (BLE) communication was implemented using a General Attribute Profile (GATT) server on the pill, with a smartphone acting as a client. Commands were sent from the client (smartphone) to the server on the pill, where they were decoded to activate or deactivate the device. The STMtool application was used for message transmission and monitoring of received radio frequency (RF) power. For power signal monitoring, the RF received was measured with the STMtool meter application. This involved moving the smartphone to three different distances, ranging from 30 to 90 cm, in direct line of sight, with the pill wrapped in a 25-mm layer of pork belly tissue. As depicted in FIG. 9C, testing showed that at lateral distances of 30 to 90 cm between the pill and a smart device, signal power attenuated from −63 to −76 dBm (500 to 25 pW), which is about four times higher than the minimum power sensibility of −82 dBm (6.3 pW) required for Bluetooth Low Energy (BLE) communication. Hence, this signal strength was sufficient for reliable monitoring and control of the robotic pill using a smartphone, tablet, or laptop at short range.

The motor driver can deliver power to the screw motor to capture mucus while monitoring the current, which is dependent on the capturing sample. An increase of 1 mA has been observed when the screw is capturing or in contact with phantom tissue/gel. By monitoring the current, the actual collection of mucus can be sensed using this current monitor as a proxy or indicator.

A power driver block is included to manage power to the motors and sense current consumption, which for the one driving the screw is closely related with the flow of the captured sample and can be used to estimate the captured sample volume.

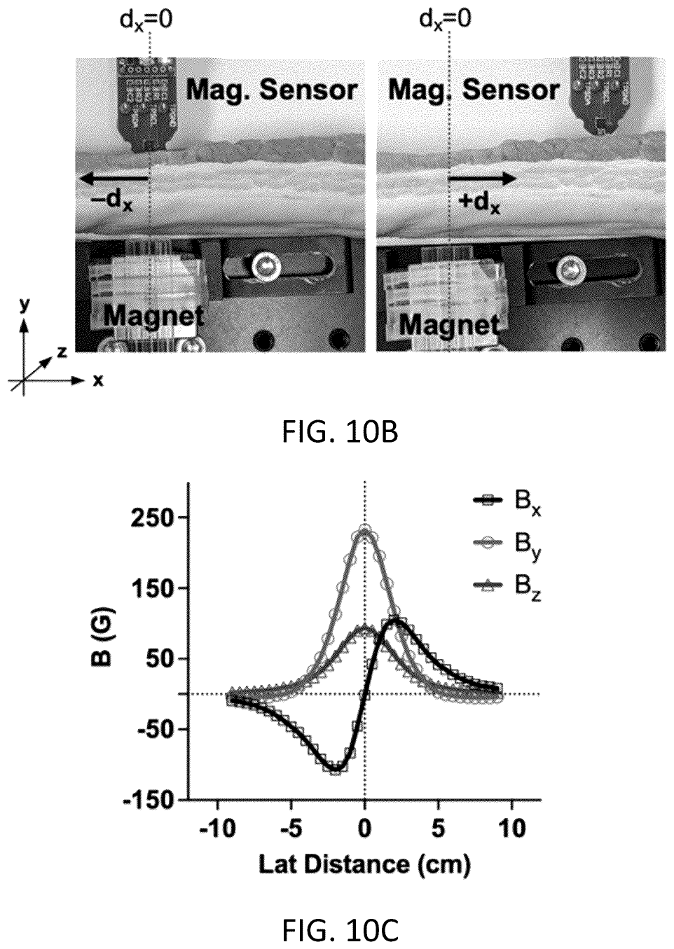

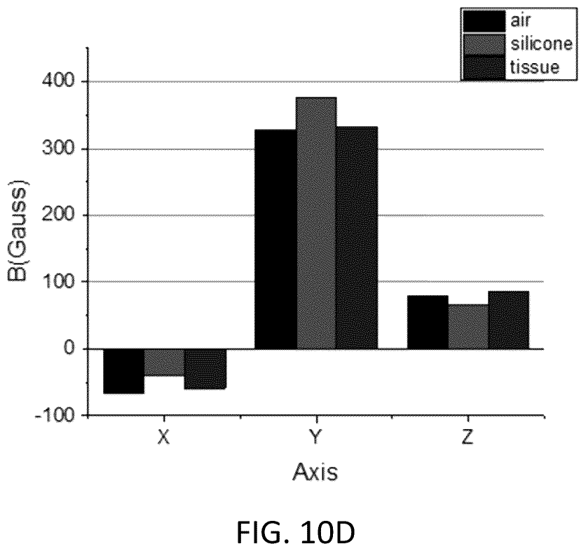

With reference being had to FIGS. 10A-10D, a three-axis (3D) Hall effect magnetic sensor is used in the location system of the pill. The sensor can communicate with the microcontroller using I2C port, sending three dimensional measurements of the magnetic field produced by an external magnet source (permanent magnet or electromagnet) for triangulation and estimation of the pill location. That is to say, in the presence of an external magnet, the pill can detect the magnitude of the magnetic field along three axes, which varies with the cube of the separation distance. This allows the system to calculate the distance and direction relative to the magnetic source. FIGS. 10B and 10C illustrate how lateral movement of the pill sensor generated variations in magnetic flux density B, which is a function of the sensor's position, as well as the geometry and magnetization of the permanent magnet. As shown in FIG. 10C, the magnetic field changes in intensity in different axis (x, y, and z) as the pill increases distance from the permanent magnet. Thus, although only the Hall effect magnetic sensor is illustrated in FIG. 10B and that sensor is apart from the robotic pill, it is apparent that such an arrangement could be used for precise location detection of a robotic pill in three-dimensional space within a body. An accelerometer/gyroscope is used to obtain additional positioning information of the pill, it communicates with the microcontroller through the I2C port and returns motion and orientation of the pill, which is used in conjunction with the magnetic sensor described previously to obtain more accurate location estimation of the pill.

pH sensing may also be included for alternate location detection of the pill. An amplifier either instrumentation or transconductance is included for reading of pH sensor through the A/D converter of the microcontroller. The GI tract has a very characteristic pH profile along the different sections of the tract that it is used in conjunction with the device transit time as an alternate/additional simple method to locate the robotic pill.

In at least some forms, an externally placed Hall effect 2D magnetic sensor array will be designed to locate the pill and determine distance from the surface via Z-axis magnetic field magnitude, producing 3D visualization and monitoring pill movement, orientation, and displacement. The body does not produce any detectable magnetic signal; the high imaging signal-to-noise ratio facilitates selectively detecting the pill magnetic core. pH data can supplement location capabilities as described above while assessing and accounting for individual variance, and mobility can be measured with an accelerometer/gyroscope to create a robust localization/identification model of the relevant sections of the GI tract to trigger sample capture in the regions of interest.

State observer control algorithms for real-time location estimation can enable sample capture when at the region of interest. An on-pill Bluetooth GATT (general attribute profile) server can interface with a smart device client for control and data logging. A lithium battery approved/certified for medical devices can power motor and electronics through a LDO (Low Dropout) regulator.

Materials may need to be cost-effective and biocompatible to justify the use of electronic pills that can be regularly administered similarly to oral pharmacotherapies. 28-mAh silver oxide batteries (728-1111-ND from Seiko Instruments) may be used with dimensions of 6.8 mm by 2.6 mm. These batteries are used in FDA approved ingestible devices. The PCB components are aligned with common electronic systems and materials used in FDA-approved ingestible systems. Surface electrodes may be gold wires (30 gauge, 14 karat). Gold is regarded as a highly biocompatible metal. The capsule material itself leverages stereolithography (SLA) three-dimensional (3D) printing of simulated polypropylene materials. This allows for reliable fabrication of microscale surface features, which can be done affordably with biocompatible SLA resins. If faster fabrication rates are needed for large device quantities, then computer numerical control (CNC) machining of polypropylene can be used instead with similar overall costs.

In situations in which a single robot be unable to collect sufficient bioanalytes for downstream analysis, the collection chamber volume might be altered, the collection duration could be increased, and/or use multiple robots serially.

According to one aspect, this device enables the untethered collection of samples in the GI tract. Exemplary samples collected can be viruses, exosomes, vesicles, drugs, microorganisms, cells, metabolites, enzymes, secretions, other analysts, polyps, and so forth.

In some forms, a screw conveyor or hydrodynamic screw may be used for collection of biological samples, viscous materials, and solids in the GI tract. This also applies to the collection of these types of samples directly from the intestinal wall.

In some forms, rotating chambers and/or multiple compartments may be used that open and close to collect from different locations in a spatiotemporally controlled manner.

In some forms, the pill can be tracked magnetically, ultrasonically or by other means.

In some forms, the pill can be wirelessly activated for sample collection initiation and finalization.

In some forms, the pill can have a single or multiple motors for different purposes including the active collection of sample, opening and closure of valves, driving of multi compartment chamber and biopsy extraction and delivery of molecules locally during or after docking or in transit to a region in the GI tract.

In some forms, a single motor may actuate multiple compartments and the collection mechanism (e.g., the screw).

In some forms, a reusable pill/capsule core with disposable and

interchangeable collection chambers may allow for easy access to the captured sample. The pill may be both reusable and/or disposable.

In some forms, there may be an in-chamber screw, the collection screw may be embedded in the disposable storage chamber. This can allow for rapid removal of the components in contact with the sample to reduce contamination.

In some forms, there may be a modular, removable chamber that can be different size, volume and functionality. The storage chamber may be easily removed and interchanged/replaced with a customized one for different volume capacities and screw configurations (including biopsy extraction), allowing the use of the main pill body independent of the chamber configuration. The pill can be designed as a multitool where it can capture mucosa or other surrounding molecules or take a biopsy by using something sharp that can cut out the tissue as a multi-function. The presence of the motor actuator allows the change of the tool from a sample collector to a biopsy cutter or puncher depending on the target that is being targeted for collection or delivery.

In some forms, there may be pH monitoring within the bodily cavities such as GI tract which is facilitated by wireless communication.

In some forms, the circuit board may allow the system to autosense its environment and use the local information to collect viscous fluids/samples locally (autonomous sensing and sampling).

In some forms, there may be data logging. There may be storage of system variables and wireless data logging for further analysis of the system and functionalities.

In some forms, the pill can actively learn its environment by machine learning/artificial intelligence and start or stop collection.

In some forms, in addition to the wireless control of the pill, there can be other modalities to actuate the device. External waves can trigger also the actuation of the pill for various functions in addition to the wireless actuation such as sound or magnetic waves to initiate the pill to perform a function. There may be generation of ultrasonic waves or other optical, electrical, electronic signals from inside out direction for imaging, sensing and other biomedical uses.

In some forms, the whole pill can be coated to protect the electronics and a coating layer such as enteric coating can be used to activate the device upon its digestion or exposure to the right environment.

Accordingly, disclosed herein is a small and compact exemplary robotic pill designed for capturing viscous fluids. The collection mechanism is based on the motorized 3D-printed small-factor hydrodynamic screw conveyor, which rapidly collected and stored up to 400 μL of viscous sample within one minute. The exemplary pill further features a circuit board integrating a wireless microcontroller and motor driver to enable remote motor operation via Bluetooth Low Energy (BLE) communication. The BLE signal remained stable and reliable within 1-meter range, even when passing through a 25-mm thick tissue barrier. Additionally, the exemplary bill is equipped with 3-axis (3D) Hall effect magnetic sensor, capable of detecting magnetic fields. This sensor measures the magnetic signal strength, which varied proportionally to the cube of the distance from the magnetic source; thus, enabling precise 3D localization of the pill. The exemplary pill can be docked or positioned in targeted regions using the magnetic forces generated between the embedded magnetic materials inside the pill and an external magnetic field. With positioning forces reaching up to 0.9 N, the exemplary robotic pill could be reliably guided and docked in the desired location to initiate the sampling of viscous fluids via the BLE communication. Moreover, successful capture of biological analytes, such as hemoglobin and proteins, was demonstrated using mock viscous samples, along with quantification of the captured samples. Expanding the ability to analyze mucus biospecimens in the future could provide critical insights into GI diseases, especially since the intestinal mucus, predominantly produced in the GI tract, harbors a rich array of biomarkers for disease detection. This could provide a practical and cost-effective platform for improved early diagnostic assessment and continuous monitoring in selected regions of the GI tract.

The current system's BLE communication range is limited to ˜1 meter, which may restrict its use without additional signal relays. If greater distances are needed, it is contemplated that a Bluetooth repeater belt could be implemented (or depending on the particular form of wireless communications, other types of repeaters), allowing a robotic pill to be operated from a remote computer within the line-of-sight limits of BLE communication, typically within 50-100 meters range. In addition, the robotic pill's magnetic actuation and localization could be affected by variable tissue densities, GI motility, or interference from surrounding materials in real biological settings. While the experiments with mock viscous samples successfully demonstrated analyte capture, the robotic pill's performance in dynamic, heterogeneous biological environments, where mucus viscosity and composition vary, may be further validated. Furthermore, the instant calculation model, while based on a simplified Newtonian and uniform fluid environment, provides a valuable foundation for understanding the robotic pill's performance. Future work may expand upon the effect of the complex rheological behavior of mucus, particularly its non-Newtonian characteristics, which presents unique challenges during sampling. For instance, the shearing action of the rotating screw may induce normal stresses, potentially influencing capsule positioning. Additionally, the stratified nature of mucus means that the robotic pill's interaction with different layers could impact sampling outcomes. Nevertheless, some of these potential complexities have been proactively addressed by incorporating magnetic docking and actuation mechanisms, enabling the robotic pill to maintain its position during sampling, thus enhancing the collection of the sample. This strategic design choice mitigates the potential for displacement due to normal stress and helps to target the desired sampling depth.

Thus, a compact and intelligent exemplary robotic pill has been disclosed above that is designed for active sampling of viscous fluids. Particularly when equipped with a motorized hydrodynamic screw, wireless BLE communication, and a 3-axis Hall effect magnetic sensor, such a robotic pill can enable efficient and site-specific collection. The magnetic docking mechanism provides enhances stability and addresses challenges associated with mucus's complex rheological properties. While in vitro evaluations are provided above to demonstrate its efficacy in capturing biological analytes, further in vivo validation will establish its performance in dynamic biological environments, including variations in mucus viscosity, GI motility, and potential signal interference. When used, in vivo, a multi-compartment chamber may be integrated to allow sample collection from different sections of the GI tract. Additional sensors are certainly contemplated and may be employed and validated, such as 1) an accelerometer/gyroscope to track the robotic pill's motion and orientation, and 2) an instrumentation amplifier to help characterize and map the GI tract, offering an alternative method to locate the pill based on pH profiles of the different GI sections. By enabling minimally invasive, repeatable sampling, this robotic pill has the potential to address the limitations of existing procedures, such as colonoscopies and imaging techniques, which are not suitable for frequent, repeated measurements.

It should be appreciated that various other modifications and variations to the preferred embodiments can be made within the spirit and scope of the invention. Therefore, the invention should not be limited to the described embodiments. To ascertain the full scope of the invention, the following claims should be referenced.

What is claimed is:

1. A robotic pill system for collecting one or more samples from human and animal body cavities and liquid resources, the system comprising:
a robotic pill adapted to collect the one or more samples when the robotic pill is received or introduced or taken through an orifice, other natural or surgical openings, or a cavity of the subject, the robotic pill including:
a housing having an opening placing an inside of the housing in fluid communication with the surrounding environment,
a screw present in the housing,
a motor to drive the screw, and
one or more collection chambers,
wherein the screw is adapted to collect the one or more samples from the opening in the housing by rotation of the screw by the motor for delivery of the one or more samples into one or more collection chambers in the housing of the robotic pill.

2. The robotic pill system of claim 1, wherein the one or more collection chambers are selectable for use in collection of the one or more samples with the screw in order to provide for the collection of various samples in a spatiotemporally-controlled manner.

3. The robotic pill system of claim 1, further comprising an external computing device, wherein the robotic pill is wirelessly in communication with the external computing device for at least one of control of the robotic pill and collection of data from the robotic pill.

4. The robotic pill system of claim 1, the robotic pill system further comprising an external magnet apart from the robotic pill, and wherein the robotic pill further comprises one or more magnets coupled to the robotic pill to facilitate a magnetic actuation and docking of the robotic pill with the external magnet, the external magnet being physically spaced from the robotic pill and not part of the robotic pill.

5. The robotic pill system of claim 1, wherein the robotic pill system further includes a three-axis Hall effect magnetic sensor, in which the three-axis Hall effect magnetic sensor is configured to measure a magnitude of a magnetic field along three axes, which varies with the separation distance from the magnet, in order to spatially locate the robotic pill.

6. The robotic pill system of claim 5, wherein the three-axis Hall effect magnetic sensor is physically coupled and integrated into the robotic pill.

7. The robotic pill system of claim 1, wherein the one or more samples include mucosal samples.

8. The robotic pill system of claim 1, wherein the robotic pill further includes a control electronic board in electrical communication with at least the motor.

9. The robotic pill system of claim 8, wherein the control electronic board includes a wireless communication interface that is in wireless communication with an external computing device.

10. The robotic pill system of claim 8, wherein the control electronic board is configured to transmit to the external computing device via the wireless communication interface information relating to a spatial positioning of the robotic pill and wherein the control electronic board is configured to receive via the wireless communication interface instructions relating to an operation of the screw by the motor.

11. A method of operating the robotic pill system of claim 1 after the robotic pill received or introduced or taken through an orifice, other natural or surgical openings, or a cavity of the subject, the method comprising:
operating the screw to transport the sample through an opening in the housing and into the one or more collection chambers to collect the sample.

12. The method of claim 11, the method further comprising, before operating the screw, positioning the robotic pill at an operation location within the subject.

13. The method of claim 12, wherein positioning the robotic pill at the operation location within the subject involves directing the location of the robotic pill by magnetically manipulating the position of the robotic pill to the operation location.

14. The method of claim 12, wherein positioning the robotic pill at the operation location within the subject involves magnetically docking the pill with an external magnet to hold the robotic pill in the operation location.

15. The method of claim 12, the method further comprising, before operating the screw, using a 3-axis Hall effect sensor to spatially locate the robotic pill.

16. The method of claim 11, wherein operating the screw to transport the sample through an opening in the housing and into the one or more collection chambers involves the robotic pill receiving instructions via a wireless communication interface to control operation of the screw by the motor.

17. The method of claim 11, wherein the sample is a mucosal sample.

18. The method of claim 11, wherein, after the sample is collected, the robotic pill is removed or otherwise exerted from the subject and recovered and the method further includes the step of recovering the sample from the robotic pill.

19. The method of claim 18, wherein after recovering the sample from the robotic pill, performing an analysis of the collected sample.
